(19) United States
(12) Patent Application Publication
(10) Pub. No.: US 2026/0098675 A1
(43) Pub. Date: Apr. 9, 2026

(54) NOVEL SELF-ABSORBENT ICE PACK

(71) Applicant: SHANGHAI HUIZHOU INDUSTRIAL CO., LTD., SHANGHAI (CN)

(72) Inventors: JUN ZHANG, SHANGHAI (CN); YIRUI DING, SHANGHAI (CN); QIANGWEN ZHANG, SHANGHAI (CN); CHENGQIANG XU, SHANGHAI (CN); MUSEN ZHOU, SHANGHAI (CN)

(21) Appl. No.: 18/923,895

(22) Filed: Oct. 23, 2024

(30) Foreign Application Priority Data
Oct. 8, 2024 (CN) 202411395658.7

(51) Int. Cl. F25D 3/00

(52) U.S. Cl. CPC F25D 3/00; F25D 2303/0822

(57) ABSTRACT

The disclosure belongs to the technical field of ice packs, and in particular to a novel self-absorbent ice pack, including: an outer bag body and absorbent resin granules filling an inside of the outer bag body. The outer bag body includes a composite plastic layer and a water-permeable layer. In this solution, a semi-composite plastic strip is arranged in a middle part of the water-permeable layer, non-woven fabric surfaces on the two sides of the semi-composite plastic strip are respectively used for water inflowing and air evacuation, which improves the water inflowing efficiency and prevents the absorbent resin granules inside from oozing out. When water enters the inside of the ice pack and the ice pack swells, the semi-composite plastic strip protrudes. The ice packs contact mainly at the protruding semi-composite plastic strips. Thus, the surface is less likely to retain water or freeze and stick.

### TECHNICAL FIELD

The disclosure relates to the technical field of ice packs, and in particular to a novel self-absorbent ice pack.

### BACKGROUND

An ice pack is a novel freezing medium, which has no water pollution when thawed and melted, and can be used repeatedly under both cold and hot conditions. Its effective cold capacity is 6 times that of the same volume of ice. It can be used instead of dry ice, ice cubes, etc. The ice pack is widely used. For example, the ice pack can be used in medical treatment, such as bringing down a high fever, diminishing inflammation and relieving pain, and adjuvant physiotherapy, such as cold compress for beauty treatment, sprain relief, hemostasis, purulency treatment and skin care. The ice pack can be used for refrigerated transportation of various cryoprotectant agents, and long-distance refrigerated transportation of solder paste, poultry drugs, medicines, plasma, vaccines, aquatic products, poultry, ornamental fish and fresh food in foreign trade. The ice pack can also be used for injuries, sprains and falls of sports athletes during training and competitions. Moreover, the ice pack can be used for cold storage for power saving in daily life, and for maintaining low temperature, keeping food fresh and cooling drinks when the refrigerator is out of power or during travel.

At present, the commonly used ice packs are as follows: FIG. 1 to FIG. 3 show an existing ice pack I. The ice pack I has a non-woven fabric surface 1 and a composite plastic layer 2. Edges of the non-woven fabric surface 1 and the composite plastic layer 2 are hot-pressed to form a heat-sealed edge I 3, which is filled with absorbent resin granules 4 inside. When in use, the ice pack I is put into water, and water permeates to the inside through the non-woven fabric surface 1. The water comes into contact with the absorbent resin granules 4, and the absorbent resin granules 4 absorb the water and swell.

FIG. 4 to FIG. 6 show an existing ice pack II. One side edge of a non-woven fabric surface 1 is connected to a semi-composite plastic strip 5 by hot pressing. In this way, when the ice pack II is immersed in water, the water enters the inside through the non-woven fabric surface 1. The semi-composite plastic strip 5 takes up most of the area. When the ice pack II swells, the semi-composite plastic strip 5 protrudes. When adjacent ice packs II are in contact, the semi-composite plastic strip 5 is less likely to retain water due to its smooth surface, and therefore, it is less susceptibility to icing and sticking.

There is also an ice pack III (not shown) formed by hot-pressing two composite plastic layers 2, and micropores are made in surfaces of the composite plastic layers 2 by laser drilling.

The above ice packs have the following problems: The non-woven fabric surface 1 of the ice pack I has a porous surface. When the ice pack I is squeezed by water, a middle part of the non-woven fabric surface 1 protrudes outwards. Since the non-woven fabric surface 1 is water-permeable, there is water residing on the surface of the non-woven fabric surface 1. In this case, when the ice packs I are frozen, the non-woven fabric surfaces 1 of the ice pack I are prone to freeze and adhere to each other. If the ice packs I are forced apart, the non-woven fabric surfaces 1 are prone to tear.

When the ice pack II is immersed in water, since the non-woven fabric surface 1 is located on one side of the semi-composite plastic strip 5, both water inflowing and air evacuation are realized through the non-woven fabric surface 1, causing low water inflowing efficiency.

The ice pack III has fewer micropores than the pores in the non-woven fabric surface 1, which leads to slow water inflowing. If a pore size of the micropores is increased, the absorbent resin granules 4 will easily ooze out.

### SUMMARY

An objective of the disclosure is to provide a novel self-absorbent ice pack to solve the problems in the Background.

In order to achieve the above objective, the disclosure provides the following technical solution: A novel self-absorbent ice pack includes:
an outer bag body and absorbent resin granules filling an inside of the outer bag body,
where the outer bag body includes a composite plastic layer and a water-permeable layer, an edge of the water-permeable layer and an edge of the composite plastic layer being hot-pressed to form a heat-sealed edge I; and
the water-permeable layer includes a non-woven fabric surface and a semi-composite plastic strip arranged in a middle part of the non-woven fabric surface, and a joint between the semi-composite plastic strip and the non-woven fabric surface is hot-pressed to form a heat-sealed edge II.

Preferably, the composite plastic layer is made of a PE material.

Preferably, a number of the non-woven fabric surfaces is set to one, and the semi-composite plastic strip is hot-pressed to the middle part of the non-woven fabric surface.

Preferably, a number of the non-woven fabric surfaces is set to two, and a hot pressing space is arranged between the two non-woven fabric surfaces, the semi-composite plastic strip being located in the hot pressing space, and edges on two sides of the semi-composite plastic strip being connected to corresponding edges of the non-woven fabric surfaces on two sides by hot pressing.

Preferably, a width of the non-woven fabric surface between the heat-sealed edge I and the semi-composite plastic strip is 2.8 cm-3.2 cm.

Preferably, the composite plastic layer and the semi-composite plastic strip are made of a same material.

Preferably, a one-cell water storage region or a multi-cell water storage region is formed between the composite plastic layer and the water-permeable layer; and when the multi-cell water storage region is formed between the composite plastic layer and the water-permeable layer, a plurality of heat-sealed edges III intersecting transversely and longitudinally are formed between the composite plastic layer and the water-permeable layer by hot pressing, and a region formed between the heat-sealed edges III is the multi-cell water storage region.

Preferably, the novel self-absorbent ice pack is specifically prepared by the following steps:
S1: setting sizes of the composite plastic layer and the water-permeable layer, and cutting the composite plastic layer;
S2: hot-pressing the non-woven fabric surface and two sides of the semi-composite plastic strip, then cutting the non-woven fabric surface and the semi-composite plastic strip as a whole according to a size to form the water-permeable layer, a size of the water-permeable layer being the same as that of the composite plastic layer; and
S3: attaching the composite plastic layer to the water-permeable layer, hot-pressing three edges of the composite plastic layer and the water-permeable layer to form an ice pack with an opening, filling the ice pack with the absorbent resin granules through the opening, and sealing the opening of the ice pack by hot pressing.

Compared with the prior art, the disclosure has the following advantages:

In this solution, the semi-composite plastic strip is arranged in the middle part of the water-permeable layer, the non-woven fabric surfaces on the two sides of the semi-composite plastic strip are respectively used for water inflowing and air evacuation, or water inflowing and air evacuation are carried out through the non-woven fabric surfaces on the two sides at the same time, which improves the water inflowing efficiency and prevents the absorbent resin granules inside from oozing out.

When water enters the inside of the ice pack and the ice pack swells, the semi-composite plastic strip protrudes. The ice packs contact mainly at the protruding semi-composite plastic strips. Thus, the surface is less likely to retain water or freeze and stick.

### BRIEF DESCRIPTION OF FIGURES

FIG. 1 is a schematic structural view of a non-woven fabric surface of an existing ice pack I;

FIG. 2 is a sectional view of the existing ice pack I;

FIG. 3 is a schematic structural view of a composite plastic layer of the existing ice pack I;

FIG. 4 is a schematic structural view of a non-woven fabric surface and a semi-composite plastic strip of an existing ice pack II;

FIG. 5 is a sectional view of the existing ice pack II;

FIG. 6 is a schematic structural view of a composite plastic layer of the existing ice pack II;

FIG. 7 is a schematic structural front view of the disclosure;

FIG. 8 is a sectional view of the disclosure;

FIG. 9 is a back view of the disclosure;

FIG. 10 is a schematic structural view of a non-woven fabric surface and a semi-composite plastic strip of the disclosure connected by a first method; and

FIG. 11 is a schematic structural view of the non-woven fabric surface and the semi-composite plastic strip of the disclosure connected by a second method.

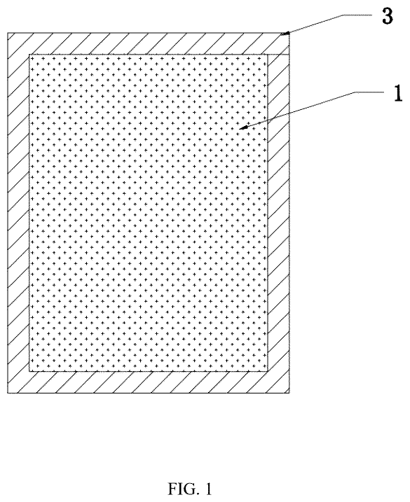

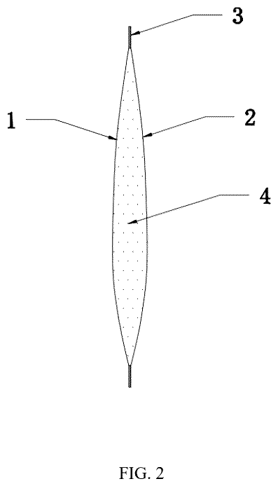

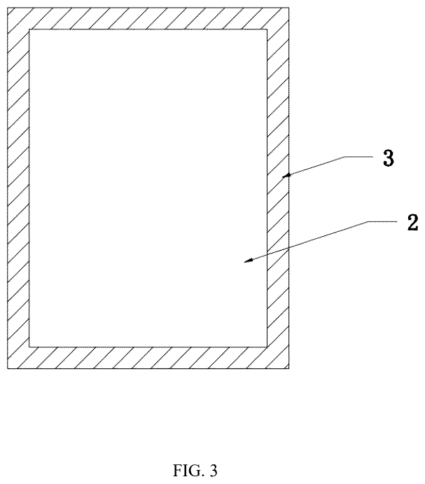

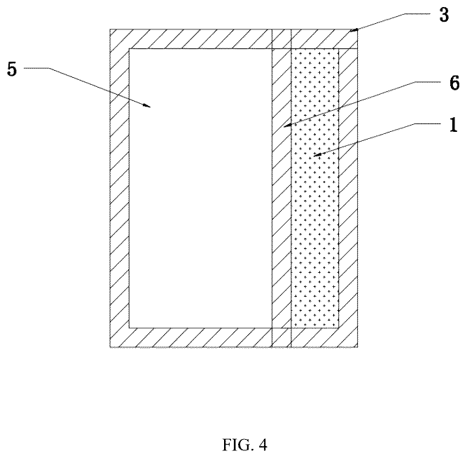

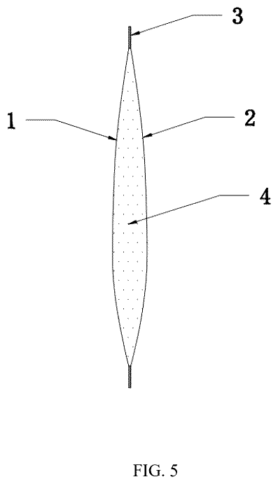

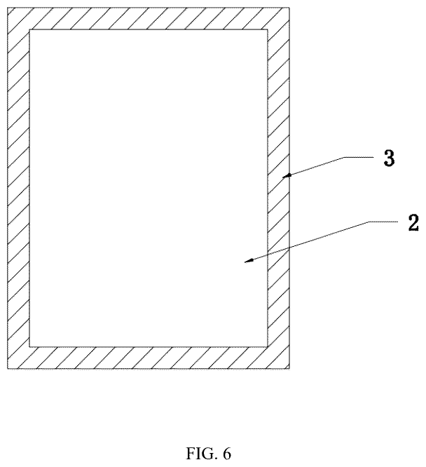

In the figures: 1. non-woven fabric surface; 2, composite plastic layer; 3, heat-sealed edge I; 4, absorbent resin granule; 5, semi-composite plastic strip; 6, heat-sealed edge II; and 7, hot pressing space.

### DETAILED DESCRIPTION

The technical solutions in embodiments of the disclosure will be clearly and completely described below with reference to the accompanying drawings in the embodiments of the disclosure. It is apparent that the described embodiments are only a part, rather than all of the embodiments of the disclosure. All other embodiments obtained by those of ordinary skill in the art based on the embodiments of the disclosure without creative work are within the protection scope of the disclosure.

In the description of the disclosure, it should be understood that, the orientation or positional relationship indicated by the term “upper”, “lower”, “front”, “rear”, “left”, “right”, “top”, “bottom”, “inner”, “outer” or the like is based on the orientation or positional relationship shown in the accompanying drawings. This is only for the convenience of describing the disclosure and simplifying the description, and does not indicate or imply that a device or element must have a specific orientation, or be constructed and operated in a specific orientation, and therefore cannot be understood as a limitation of the disclosure.

### EMBODIMENT I

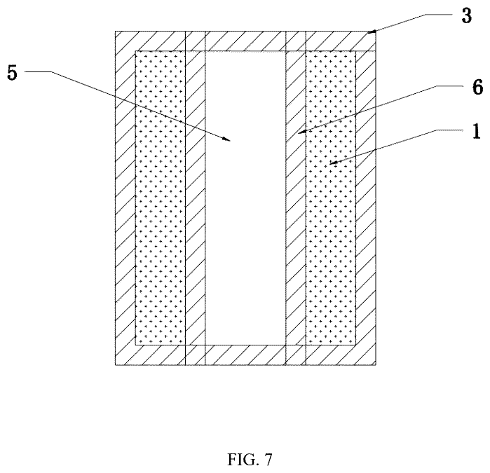

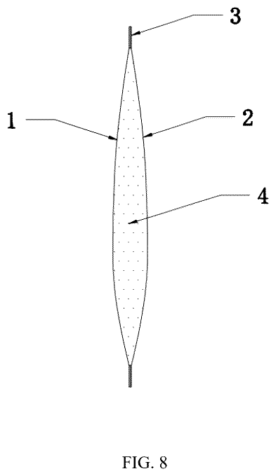

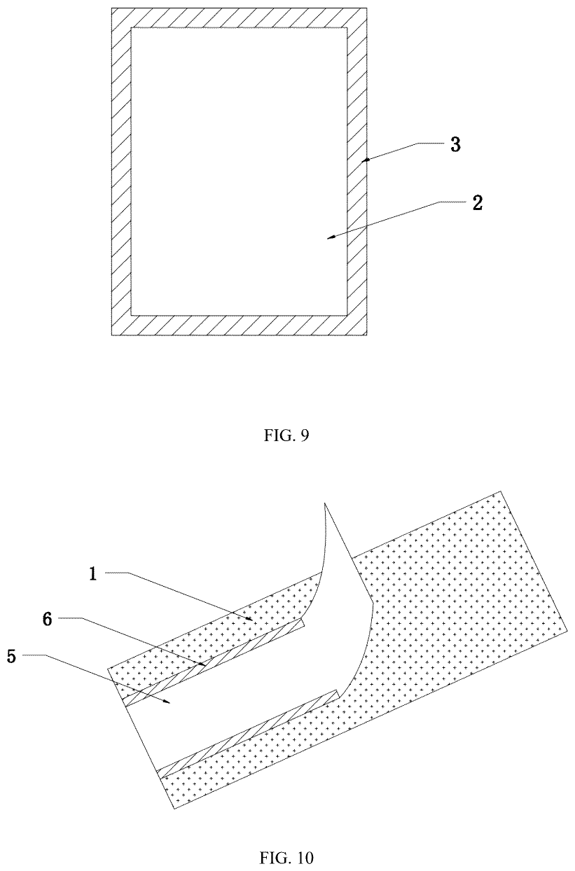

Referring to FIG. 7 to FIG. 11, the disclosure provides a technical solution: A novel self-absorbent ice pack includes:
an outer bag body and absorbent resin granules 4 filling an inside of the outer bag body.

The outer bag body includes a composite plastic layer 2 and a water-permeable layer. An edge of the water-permeable layer and an edge of the composite plastic layer 2 are hot-pressed to form a heat-sealed edge I 3. The water-permeable layer includes a non-woven fabric surface 1 and a semi-composite plastic strip 5 arranged in a middle part of the non-woven fabric surface 1. A joint between the semi-composite plastic strip 5 and the non-woven fabric surface 1 is hot-pressed to form a heat-sealed edge II 6.

Analysis for the above contents: When in actual use, the ice pack is put in water, with the non-woven fabric surface 1 on one side below the water surface and the non-woven fabric surface 1 on the other side above the water surface. In this way, the non-woven fabric surface 1 below the water surface can be used for water inflowing, and the non-woven fabric surface 1 on the other side can be used for air evacuation, so that air and water flow along a single direction without hindering each other. Thereby, the overall water inflowing efficiency is higher. When enough water enters the ice pack, the whole ice pack swells up. Since the semi-composite plastic strip 5, which is located in the middle part, protrudes the highest, the semi-composite plastic strips 5 of the adjacent ice packs are most likely to be close to each other. Due to material characteristics of the semi-composite plastic strip 5, its surface is smooth and thus it is not easy to freeze and stick between the adjacent ice packs.

### EMBODIMENT II

Referring to FIG. 7 to FIG. 11, the disclosure provides a technical solution based on Embodiment I: The composite plastic layer 2 is made of a PE material. The composite plastic layer 2 and the semi-composite plastic strip 5 are made of a same material.

Analysis for the above contents: The PE material has a smooth surface and good toughness, and is not easy to get wet.

### EMBODIMENT III

Referring to FIG. 7 to FIG. 11, the disclosure provides a technical solution based on Embodiment I: The non-woven fabric surface 1 and the semi-composite plastic strip 5 are connected by the following two methods:

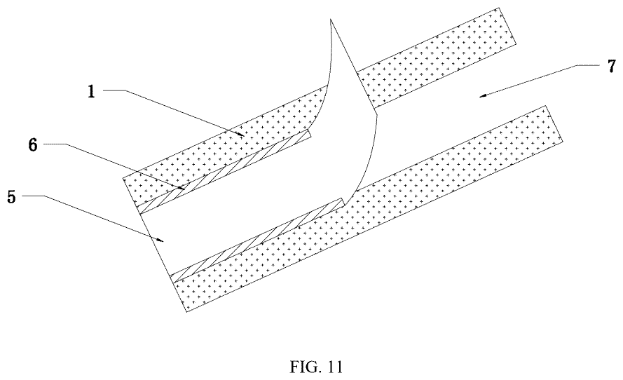

First method: As shown in FIG. 10, a number of the non-woven fabric surfaces 1 is set to one, and the semi-composite plastic strip 5 is hot-pressed to the middle part of the non-woven fabric surface 1.

Second method: As shown in FIG. 11, a number of the non-woven fabric surfaces 1 is set to two, and a hot pressing space 7 is arranged between the two non-woven fabric surfaces 1. The semi-composite plastic strip 5 is located in the hot pressing space 7, and edges on two sides of the semi-composite plastic strip 5 are connected to corresponding edges of the non-woven fabric surfaces 1 on two sides by hot pressing.

Analysis for the above contents: Both the two methods for connecting the non-woven fabric surface 1 and the semi-composite plastic strip 5 can be achieved. The second method is relatively material-saving, and the first method is relatively simple to implement.

### EMBODIMENT IV

Referring to FIG. 7 to FIG. 11, the disclosure provides a technical solution based on Embodiment III: A width of the non-woven fabric surface 1 between the heat-sealed edge I 3 and the semi-composite plastic strip 5 is 2.8 cm-3.2 cm.

Analysis for the above contents: The width of the non-woven fabric surface 1 between the heat-sealed edge I 3 and the semi-composite plastic strip 5 is preferably 3 cm, which can ensure the water absorption efficiency and avoid adhesion.

### EMBODIMENT V

Referring to FIG. 7 to FIG. 11, the disclosure provides a technical solution based on Embodiment I: A one-cell water storage region or a multi-cell water storage region is formed between the composite plastic layer 2 and the water-permeable layer. When the multi-cell water storage region is formed between the composite plastic layer 2 and the water-permeable layer, a plurality of heat-sealed edges III intersecting transversely and longitudinally are formed between the composite plastic layer 2 and the water-permeable layer by hot pressing, and a region formed between the heat-sealed edges III is the multi-cell water storage region.

Analysis for the above contents: When the whole ice pack is made with a large size, in order to make the absorbent resin granules 4 dispersed uniformly, the inside of the ice pack is divided into a plurality of cells (water storage region), so that the absorbent resin granules 4 can be dispersed in the plurality of cells. The absorbent resin granules in the plurality of cells can absorb water separately. Moreover, the cells are isolated by the heat-sealed edges III, and thus, are less prone to affect each other.

### EMBODIMENT VI

Referring to FIG. 7 to FIG. 11, the disclosure provides a technical solution based on Embodiment I: The novel self-absorbent ice pack is specifically prepared by the following steps:
S1: setting sizes of the composite plastic layer 2 and the water-permeable layer, and cutting the composite plastic layer 2;
S2: hot-pressing the non-woven fabric surface 1 and two sides of the semi-composite plastic strip 5, then cutting the non-woven fabric surface 1 and the semi-composite plastic strip 5 as a whole according to a size to form the water-permeable layer, a size of the water-permeable layer being the same as that of the composite plastic layer 2; and
S3: attaching the composite plastic layer 2 to the water-permeable layer, hot-pressing three edges of the composite plastic layer 2 and the water-permeable layer to form an ice pack with an opening, filling the ice pack with the absorbent resin granules 4 through the opening, and sealing the opening of the ice pack by hot pressing.

The basic principles, main features and advantages of the disclosure have been shown and described above. For those skilled in the art, it is apparent that the disclosure is not limited to the details of the above exemplary embodiments, and the disclosure can be implemented in other specific forms without departing from the spirit or basic features of the disclosure. Therefore, from any point of view, the embodiments should be regarded as exemplary and non-limiting. The scope of the disclosure is defined by the appended claims rather than the description above, and therefore, it is intended that all changes falling within the meaning and scope of equivalent elements of the claims are included in the disclosure, and any reference numerals in the claims should not be regarded as limiting the claims involved.

Although the embodiments of the disclosure have been shown and described, for those of ordinary skill in the art, it can be understood that various changes, modifications, substitutions and variations can be made to these embodiments without departing from the principle and spirit of the disclosure, and the scope of the disclosure is defined by the appended claims and their equivalents.

What is claimed is:

1. A novel self-absorbent ice pack, comprising:
an outer bag body and absorbent resin granules (4) filling an inside of the outer bag body,
wherein the outer bag body comprises a composite plastic layer (2) and a water-permeable layer, an edge of the water-permeable layer and an edge of the composite plastic layer (2) being hot-pressed to form a heat-sealed edge I (3); and
the water-permeable layer comprises a non-woven fabric surface (1) and a semi-composite plastic strip (5) arranged in a middle part of the non-woven fabric surface (1), and a joint between the semi-composite plastic strip (5) and the non-woven fabric surface (1) is hot-pressed to form a heat-sealed edge II (6).

2. The novel self-absorbent ice pack according to claim 1, wherein the composite plastic layer (2) is made of a PE material.

3. The novel self-absorbent ice pack according to claim 1, wherein a number of the non-woven fabric surfaces (1) is set to one, and the semi-composite plastic strip (5) is hot-pressed to the middle part of the non-woven fabric surface (1).

4. The novel self-absorbent ice pack according to claim 1, wherein a number of the non-woven fabric surfaces (1) is set to two, and a hot pressing space (7) is arranged between the two non-woven fabric surfaces (1), the semi-composite plastic strip (5) being located in the hot pressing space (7), and edges on two sides of the semi-composite plastic strip (5) being connected to corresponding edges of the non-woven fabric surfaces (1) on two sides by hot pressing.

5. The novel self-absorbent ice pack according to claim 3, wherein a width of the non-woven fabric surface (1) between the heat-sealed edge I (3) and the semi-composite plastic strip (5) is 2.8 cm-3.2 cm.

6. The novel self-absorbent ice pack according to claim 4, wherein a width of the non-woven fabric surface (1) between the heat-sealed edge I (3) and the semi-composite plastic strip (5) is 2.8 cm-3.2 cm.

7. The novel self-absorbent ice pack according to claim 1, wherein the composite plastic layer (2) and the semi-composite plastic strip (5) are made of a same material.

8. The novel self-absorbent ice pack according to claim 1, wherein a one-cell water storage region or a multi-cell water storage region is formed between the composite plastic layer (2) and the water-permeable layer; and when the multi-cell water storage region is formed between the composite plastic layer (2) and the water-permeable layer, a plurality of heat-sealed edges III intersecting transversely and longitudinally are formed between the composite plastic layer (2) and the water-permeable layer by hot pressing, and a region formed between the heat-sealed edges III is the multi-cell water storage region.

9. The novel self-absorbent ice pack according to claim 1, wherein the novel self-absorbent ice pack is specifically prepared by the following steps:
S1: setting sizes of the composite plastic layer (2) and the water-permeable layer, and cutting the composite plastic layer (2);
S2: hot-pressing the non-woven fabric surface (1) and two sides of the semi-composite plastic strip (5), then cutting the non-woven fabric surface (1) and the semi-composite plastic strip (5) as a whole according to a size to form the water-permeable layer, a size of the water-permeable layer being the same as that of the composite plastic layer (2); and
S3: attaching the composite plastic layer (2) to the water-permeable layer, hot-pressing three edges of the composite plastic layer (2) and the water-permeable layer to form an ice pack with an opening, filling the ice pack with the absorbent resin granules (4) through the opening, and scaling the opening of the ice pack by hot pressing.
